(19) United States
(12) Patent Application Publication
(10) Pub. No.: US 2024/0170161 A1
(43) Pub. Date: May 23, 2024

(54) METHOD FOR PROCESSING MEDICAL DATA, APPARATUS, AND STORAGE MEDIUM

(71) Applicant: BOE Technology Group Co., Ltd., Beijing (CN)

(72) Inventors: Xue Chen, Beijing (CN); Jinnv Liu, Beijing (CN); Yude Li, Beijing (CN); Xiying Zhang, Beijing (CN); Zhenzhong Zhang, Beijing (CN); Li Zhou, Beijing (CN)

(73) Assignee: BOE Technology Group Co., Ltd., Beijing (CN)

(21) Appl. No.: 18/282,018

(22) PCT Filed: Jan. 4, 2023
(86) PCT No.: PCT/CN2023/070403

§ 371 Date: Sep. 14, 2023

(30) Foreign Application Priority Data
Feb. 28, 2022 (CN) 202210190188.5

(51) Int. Cl. G16H 50/70; G06F 18/25; G06F 40/30; G16H 50/20; G16H 50/30

(52) U.S. Cl. CPC G16H 50/70; G06F 18/25; G06F 40/30; G16H 50/20; G16H 50/30

(57) ABSTRACT

A method for processing medical data, an apparatus and a storage medium, the method includes: acquiring a case-history datum, and performing a target process to obtain a disease-analysis vector, wherein the target process includes: generating a case-history semantic vector of the case-history datum; for each of preset diseases in a preset-disease set, determining a first possibility weight of the case-history datum caused by the preset disease according to the case-history semantic vector, to obtain a first weight vector; according to case-history symptoms and case-history diseases in the case-history datum, determining from a predetermined knowledge graph a candidate disease that is capable of generating generate the case-history datum; determining a second possibility weight of the case-history datum caused by the candidate disease, to obtain a second weight vector; and fusing the first weight vector and the second weight vector, to obtain the disease-analysis vector corresponding to the case-history datum.

### CROSS REFERENCE TO RELEVANT APPLICATIONS

The present application claims the priority of the Chinese patent application filed on Feb. 28, 2022 before the China National Intellectual Property Administration with the application number of 202210190188.5 and the title of “MEDICAL DATA PROCESSING METHOD AND APPARATUS, AND STORAGE MEDIUM”, which is incorporated herein in its entirety by reference.

### TECHNICAL FIELD

The present disclosure relates to the technical field of data processing, and particularly relates to a method for processing medical data, an apparatus and a storage medium.

### BACKGROUND

With the development of the medical technology, the automated analysis and processing on medical data is increasingly required in medical education, the construction of electronic medical interrogation platforms, and so on, so as to satisfy different demands on analysis.

Currently, because of the particularity of the medical data, the processing on the medical data still mainly relies on artificial processing. However, the artificial processing has an extremely low efficiency, and, especially in the scenes that have demands on the processing and analysis on a large quantity of data, such as electronic medical interrogation platforms and analytical statistics of medical data, it is still of a very large difficulty in the increase of the efficiency of the processing on the medical data.

### SUMMARY

The present disclosure provides a method for processing medical data, wherein the method includes:
acquiring a case-history datum, and performing a target process to obtain a disease-analysis vector corresponding to the case-history datum, wherein the target process includes:
generating a case-history semantic vector of the case-history datum;
for each of preset diseases in a preset-disease set determining a first possibility weight of the case-history datum caused by the preset disease according to the case-history semantic vector, to obtain a first weight vector;
according to case-history symptoms and case-history diseases in the case-history datum, determining from a predetermined knowledge graph a candidate disease that is capable of generating generate the case-history datum, wherein the predetermined knowledge graph includes entities and relations that are relevant to the preset disease, and the candidate disease belongs to the preset-disease set:
determining a second possibility weight of the case-history datum caused by the candidate disease, to obtain a second weight vector; and
fusing the first weight vector and the second weight vector, to obtain the disease-analysis vector corresponding to the case-history datum.

Optionally, the case-history datum includes a text datum and a numerical-value datum, and the step of generating the case-history semantic vector of the case-history datum includes:
encoding the text datum into a text semantic vector:
converting the numerical-value datum into a vector, to obtain a numerical-value vector:
stitching the text semantic vector and the numerical-value vector, to obtain a stitched vector; and
by using a multihead self-attention mechanism, encoding the stitched vector, to obtain the case-history semantic vector of the case-history datum.

Optionally, the step of, according to the case-history symptoms and the case-history diseases in the case-history datum, determining from the predetermined knowledge graph the candidate disease that is capable of generating the case-history datum includes:
for each of the case-history symptoms in the case-history datum, determining a graph disease subset corresponding to the case-history symptom in the predetermined knowledge graph, to form a graph-disease set; and
according to a case-history-disease set formed by the case-history diseases in the case-history datum, and the graph-disease set, determining a candidate-disease set that is capable of generating the case-history datum.

Optionally, the step of, according to the case-history-disease set formed by the case-history diseases in the case-history datum, and the graph-disease set, determining the candidate-disease set that is capable of generating the case-history datum includes:
for each of the case-history diseases in the case-history-disease set, if a negative-factor coefficient of the case-history disease is a preset minimum value, deleting the case-history disease from the case-history-disease set, wherein the case-history-disease set obtained after the case-history disease is deleted forms a target-case-history-disease set;
for each of the case-history diseases in the case-history-disease set, if the negative-factor coefficient of the case-history disease is the preset minimum value, and the case-history disease exists in the graph-disease set, deleting the case-history disease from the graph-disease set, wherein the graph-disease set obtained after the case-history disease is deleted forms an initial candidate-disease set; and
for each of target case-history diseases included by the target-case-history-disease set, if the target case-history disease does not exist in the initial candidate-disease set, adding the target case-history disease into the initial candidate-disease set, to form the candidate-disease set that is capable of generating the case-history datum.

Optionally, the step of determining the second possibility weight of the case-history datum caused by the candidate disease, to obtain the second weight vector includes:
according to the negative-factor coefficient of each of the case-history symptoms, a probability of joint occurrence of each of the case-history symptoms and the candidate disease, a quantity of diseases in the graph disease subset to which the candidate disease belongs, and a quantity of diseases in the candidate-disease set, determining an initial second possibility weight of the case-history datum caused by the candidate disease;
if the candidate disease satisfies a preset condition, determining the initial second possibility weight corresponding to the candidate disease to be the second possibility weight corresponding to the candidate disease, wherein the preset condition refers to that the candidate disease exists in the initial candidate-disease set but does not exist in the case-history-disease set; and
if the candidate disease does not satisfy the preset condition, correcting the initial second possibility weight corresponding to the candidate disease, to obtain the second possibility weight corresponding to the candidate disease.

Optionally, the step of, if the candidate disease does not satisfy the preset condition, correcting the initial second possibility weight corresponding to the candidate disease, to obtain the second possibility weight corresponding to the candidate disease includes:
if the candidate disease exists in both of the case-history-disease set and the initial candidate-disease set, according to a probability of occurrence of the candidate disease, correcting the initial second possibility weight corresponding to the candidate disease, to obtain the second possibility weight corresponding to the candidate disease; and
if the candidate disease exists in the case-history-disease set but does not exist in the initial candidate-disease set, according to the negative-factor coefficient of the candidate disease, a preset hyper-parameter and the quantity of the diseases in the candidate-disease set, correcting the initial second possibility weight corresponding to the candidate disease, to obtain the second possibility weight corresponding to the candidate disease.

Optionally, before the step of, according to the case-history symptoms and the case-history diseases in the case-history datum, determining from the predetermined knowledge graph the candidate disease that is capable of generating generate the case-history datum, the method further includes:
according to a degree of negation to the case-history symptom by a first neighboring word located at a position preceding the case-history symptom in the case-history datum, determining the negative-factor coefficient of the case-history symptom, wherein the negative-factor coefficient of the case-history symptom is negatively correlated with the degree of negation to the case-history symptom by the first neighboring word; and
according to a degree of negation to the case-history disease by a second neighboring word located at a position preceding the case-history disease in the case-history datum, determining the negative-factor coefficient of the case-history disease, wherein the negative-factor coefficient of the case-history disease is negatively correlated with the degree of negation to the case-history disease by the second neighboring word.

Optionally, after the step of, if the candidate disease does not satisfy the preset condition, correcting the initial second possibility weight corresponding to the candidate disease, to obtain the second possibility weight corresponding to the candidate disease, the method further includes:
for a preset disease that does not belong to the candidate-disease set, determining the second possibility weight corresponding to the preset disease to be 0; and
performing normalization processing to the second possibility weight corresponding to each of the preset diseases, to obtain the second weight vector.

Optionally, before the step of determining the second possibility weight of the case-history datum caused by the candidate disease, to obtain the second weight vector, the method further includes:
acquiring from the predetermined knowledge graph a probability of joint occurrence of each of the case-history symptoms and the candidate disease.

Optionally, before the step of, if the candidate disease does not satisfy the preset condition, correcting the initial second possibility weight corresponding to the candidate disease, to obtain the second possibility weight corresponding to the candidate disease, the method further includes:
acquiring from the predetermined knowledge graph a probability of occurrence of each of the candidate diseases.

Optionally, before the step of, according to the case-history symptoms and the case-history diseases in the case-history datum, determining from the predetermined knowledge graph the candidate disease that is capable of generating the case-history datum, the method further includes:
performing entity identification to the case-history datum, to obtain entity references in the case-history datum;
performing entity linking to the entity references in the predetermined knowledge graph, to obtain matched entities in the predetermined knowledge graph of the entity references:
screening out from the matched entities symptom entities that characterize symptoms, to obtain the case-history symptoms of the case-history datum; and
screening out from the matched entities disease entities that characterize diseases, to obtain the case-history diseases of the case-history datum.

Optionally, the step of performing entity linking to the entity references in the predetermined knowledge graph, to obtain the matched entity in the predetermined knowledge graph of the entity references includes:
for each of the entities included by the predetermined knowledge graph, calculating similarities between the entity references and each of the entities; and
linking the entity references to a target entity corresponding to a largest similarity of the similarities, to use the target entity as the matched entity in the predetermined knowledge graph of the entity references.

Optionally, the step of calculating the similarities between the entity references and each of the entities includes:
for any one of the entities calculating initial similarities between the entity references and the entity by using at least two similarity calculating modes; and
calculating an average value of the initial similarities that are obtained by calculation, to obtain a similarity between the entity references and the entity.

Optionally, the initial similarities include at least two of an edit-distance similarity, a Jaccard similarity, a longest-common-substring similarity, a cosine similarity, an explicit-semantic-analysis similarity and a deep-learning similarity.

Optionally, the step of performing entity identification to the case-history datum, to obtain the entity references in the case-history datum includes:
performing entity identification to the case-history datum according to a predetermined dictionary containing a plurality of entity names, to obtain the entity references in the case-history datum.

Optionally, the step of, performing entity identification to the case-history datum according to the predetermined dictionary containing the plurality of entity names, to obtain the entity references in the case-history datum includes:
according to the predetermined dictionary including the plurality of entity names, performing entity identification to the case-history datum by using a bidirectional maximum matching algorithm, to obtain the entity references in the case-history datum.

Optionally, the first weight vector and the second weight vector have equal dimensionalities, the dimensionalities are a quantity of diseases in the preset-disease set, and the step of fusing the first weight vector and the second weight vector, to obtain the disease-analysis vector corresponding to the case-history datum includes:
weighting the first possibility weight and the second possibility weight with the equal dimensionalities by using different preset importance coefficients, to obtain weighted parameters, wherein a preset importance coefficient corresponding to the first possibility weight and a preset importance coefficient corresponding to the second possibility weight are negatively correlated; and
calculating the weighted parameters by using a linear function or a nonlinear function, to obtain fused weights, wherein the fused weights form the disease-analysis vector corresponding to the case-history datum, and the disease-analysis vector has a dimensionality equal to the dimensionalities of the first weight vector and the second weight vector.

Optionally, the step of performing the target process to obtain the disease-analysis vector corresponding to the case-history datum includes:
inputting the case-history datum into a predetermined analyzing model, so that the predetermined analyzing model performs the target process, and outputs the disease-analysis vector corresponding to the case-history datum; and
before the step of acquiring the case-history datum, the method further includes:
acquiring a case-history-datum training set and a case-history-datum test set;
according to the case-history-datum training set and a predetermined loss function, training an original analyzing model, to obtain an intermediate analyzing model; and
testing the intermediate analyzing model according to the case-history-datum test set, to obtain the predetermined analyzing model.

The present disclosure further provides an apparatus for predicting a diabetes complication, wherein the apparatus includes a processor, a memory and a program stored in the memory and executable in the processor, and the program, when executed by the processor, implements the steps of the method for processing medical data stated above, to obtain the disease-analysis vector corresponding to the case-history datum, wherein the preset-disease set includes one or more diabetes complications, and each of components of the disease-analysis vector represents an illness probability corresponding to each of the diabetes complications.

The present disclosure further provides a non-transitory computer-readable storage medium, wherein an instruction in the storage medium, when executed by a processor of an electronic device, enables the electronic device to implement the method for processing medical data stated above.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

In order to more clearly illustrate the technical solutions of the embodiments of the present disclosure or the related art, the figures that are required to describe the embodiments or the related art will be briefly described below. Apparently, the figures that are described below are embodiments of the present disclosure, and a person skilled in the art can obtain other figures according to these figures without paying creative work.

FIG. 1 shows a flow chart of the steps of a method for processing medical data according to an embodiment of the present disclosure;

FIG. 2 shows a case-history datum according to an embodiment of the present disclosure;

FIG. 3 shows part of knowledge in a diabetes knowledge graph according to an embodiment of the present disclosure;

FIG. 4 shows a flow chart of the process of training a model for implementing a method for processing medical data according to an embodiment of the present disclosure;

FIG. 5 shows a flow chart of the process of using a model for implementing a method for processing medical data according to an embodiment of the present disclosure:

FIG. 6 shows a block diagram of the architecture of a predetermined analyzing model according to an embodiment of the present disclosure; and

FIG. 7 shows a flow chart of vector stitching according to an embodiment of the present disclosure.

### DETAILED DESCRIPTION

In order to make the objects, the technical solutions and the advantages of the embodiments of the present disclosure clearer, the technical solutions of the embodiments of the present disclosure will be clearly and completely described below with reference to the drawings of the embodiments of the present disclosure. Apparently, the described embodiments are merely certain embodiments of the present disclosure, rather than all of the embodiments. All of the other embodiments that a person skilled in the art obtains on the basis of the embodiments of the present disclosure without paying creative work fall within the protection scope of the present disclosure.

Unless defined otherwise, the technical terminologies or scientific terminologies used in the present disclosure should have the meanings generally understood by a person skilled in the art of the present disclosure. The words used herein such as “first” and “second” do not indicate any sequence, quantity or priority, but are merely intended to distinguish different components. Likewise, the words such as “a”, “an” or “the” do not indicate quantitative limitations, but indicate the existence of at least one instance. The words such as “comprise” or “include” mean that the element or article preceding the word encompasses the elements or articles and the equivalents thereof that are listed subsequent to the word, but do not exclude other elements or articles. The words such as “connect” or “couple” are not limited to physical or mechanical connections, but may include electric connections, regardless of direct connections or indirect connections. The orientation words such as “upper”, “lower”, “left” and “right” are merely intended to indicate relative positions on the basis of the figures, and if the absolute position of the described item has changed, the relative positions might also be correspondingly changed.

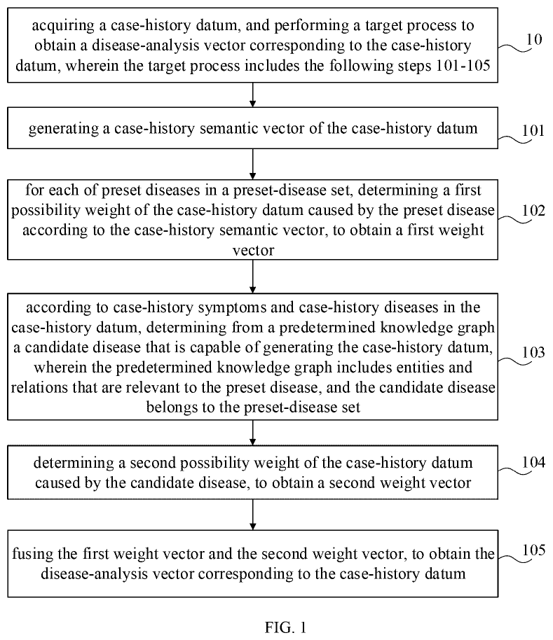

FIG. 1 shows a flow chart of the steps of a method for processing medical data according to an embodiment of the present disclosure. Referring to FIG. 1, the method includes the following steps:

Step 10: acquiring a case-history datum, and performing a target process to obtain a disease-analysis vector corresponding to the case-history datum, wherein the target process includes the following steps 101-105.

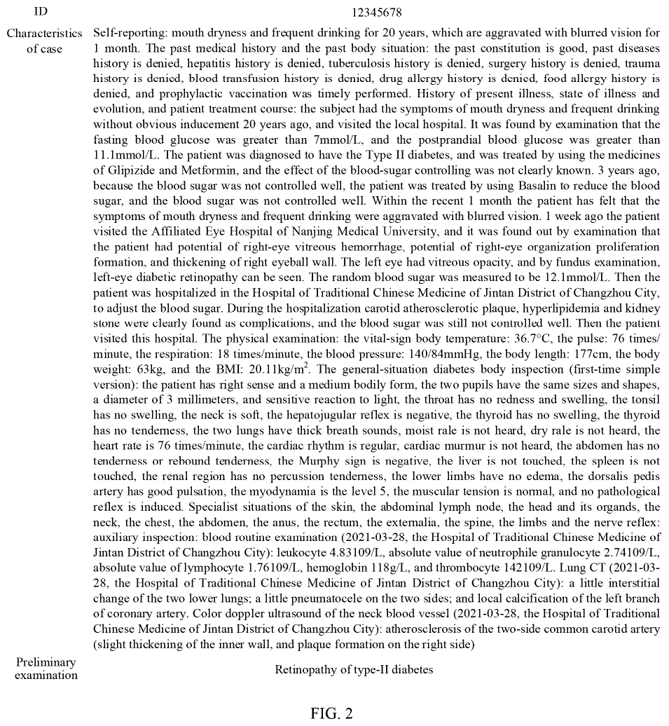

In an embodiment of the present disclosure, firstly a case-history datum to be processed may be acquired, wherein the case-history datum is an electronic datum. FIG. 2 illustratively shows a case-history datum. Optionally, the case-history datum may be obtained by converting a paper case-history into an electronic datum by means of text identification and so on. Further optionally, the case-history datum may also be obtained by data exporting of an electronic case-history datum that was directly typed into an electronic case-history platform of a hospital by a doctor during interrogation. The mode of acquiring the case-history datum is not particularly limited in the embodiments of the present disclosure.

In practical applications, for case-history data that cannot be directly processed, some pre-processing may be performed according to processing demands. The pre-processing may include anonymization processing, abnormal-data detection, structurization processing and so on. The anonymization processing can hide the privacy data of the patient such as the name, thereby protecting the privacy of the patient. The abnormal-data detection can detect the missing data and the data of abnormal numerical values in the case-history datum, and output a relevant prompt, to indicate that the case-history datum cannot be analyzed due to data abnormality. The structurization processing can convert the case-history datum into a structuralized datum, so as to facilitate the data storage, the extraction of part of the datum, and so on.

Certainly, the mode of filling in the case history may be normalized during the interrogation by the doctor, to obtain a case-history datum that can be directly processed. For example, the rule of filling in the case history may be normalized in the electronic case-history platform of the hospital, for example, providing which data must be filled in, the data of which positions should not lack, the data of which positions may be defined with the minimum values and the maximum values that can be filled in, and so on.

Step 101: generating a case-history semantic vector of the case-history datum.

In the target process, a case-history semantic vector of the case-history datum is first generated by using the semantic-analysis technique. In an embodiment of the present disclosure, the case-history semantic vector is the semantic expression of the texts and the numerical values in the case-history datum.

Step 102: for each of preset diseases in a preset-disease set, according to the case-history semantic vector, determining a first possibility weight of the case-history datum caused by the preset disease, to obtain a first weight vector.

In an embodiment of the present disclosure, the relations between the case-history datum and some diseases may be analyzed. Particularly, at least two diseases may be predetermined, to construct a preset-disease set. For example, 8 commonly seen diabetes complications (diabetic retinopathy, diabetic nephropathy, diabetic peripheral neuropathy, diabetic autonomic neuropathy, diabetic foot, atherosclerosis, diabetic peripheral vasculopathy and diabetic gastroparesis) are constructed into a preset-disease set.

Further, this step may include, based on the result of the semantic analysis of the case-history datum, calculating the possibility of incurring of the state of illness corresponding to the case-history datum by each of the preset diseases in the preset-disease set, to obtain at least two first possibility weights, and subsequently converting the first possibility weights into the required vector forms, to obtain the first weight vector.

Step 103: according to case-history symptoms and case-history diseases in the case-history datum, determining from a predetermined knowledge graph a candidate disease that might generate the case-history datum, wherein the predetermined knowledge graph contains entities and relations that are relevant to the preset disease, and the candidate disease belongs to the preset-disease set.

The case-history datum may include the data such as the past medical history and the self-reported symptoms stated by the patient himself, and the data such as the checking results and the diagnostic results of the hospital. Those data refer to various symptoms and diseases. The symptoms and the diseases that are described in the case-history datum are referred to herein as the case-history symptoms and the case-history diseases respectively.

In an embodiment of the present disclosure, a knowledge graph may be introduced, to determine the candidate diseases that might cause the state of illness corresponding to the case-history datum from another aspect. The knowledge graph may be constructed by using knowledge in the mode of a multi-relation diagram, wherein the knowledge mainly uses entity (also referred to as the head entity)-relation-entity (also referred to as the tail entity) as the smallest unit. For example, regarding redness and swelling (the symptom entity) versus (pointing to) diabetic foot (the disease entity), the relation is that the former is the symptom of the latter.

Table 1 provides an example of the entities and the relations in the knowledge graph. It can be understood that the entities and the relations in the table do not limit the present disclosure.

TABLE 1

| relation type | entity type | entity-relation example |
|---|---|---|
| anatomical structure required by | anatomical | renal tubule-Anatomy of |
| disease (Anatomy of Disease) | structure, disease | Disease-diabetic nephropathy |
| medicine required by disease (Drug | medicine, disease | RAS inhibitor-Drug of |
| of Disease) | | Disease-diabetic nephropathy |
| symptom corresponding to disease | symptom, disease | redness and swelling-Symptom of |
| (Symptom of Disease) | | Disease-diabetic foot |
| inspection item required by disease | inspection item, | hemoglobin-Test of |
| (Test of Disease) | disease | Disease-diabetic foot |
| intervention means corresponding to | intervention | amputation-Treatment of |
| disease (Treatment of Disease) | means, disease | Disease-diabetic foot |
| . . . | . . . | . . . |

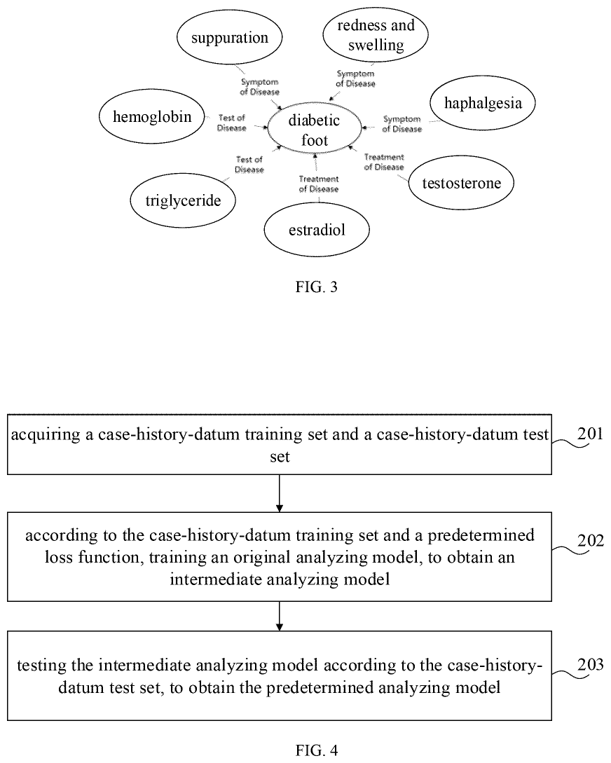

FIG. 3 illustratively shows part of knowledge in a diabetes knowledge graph, which represents the relations between the entities in the mode of a relation diagram.

In an embodiment of the present disclosure, the predetermined knowledge graph contains entities and relations that are relevant to the preset disease, wherein the relations include, for example, the symptoms, the checking means, the treatment means and the treatment medicines. The particular symptoms and disease names may be used as the entities in the predetermined knowledge graph, and the relations between them are constructed. The knowledge in the predetermined knowledge graph relevant to the preset diseases is obtained based on a large quantity of documents by means of big-data analysis and so on, and is a set of common knowledge.

In this step, the candidate diseases that might cause the state of illness corresponding to the case-history datum may be determined according to the various symptoms and diseases referred to in the case-history datum by using the common knowledge in the predetermined knowledge graph. Because the predetermined knowledge graph contains the common knowledge relevant to the preset diseases, all of the candidate diseases that are determined based on the predetermined knowledge graph belong to the preset-disease set.

Step 104: determining a second possibility weight of the case-history datum caused by the candidate disease, to obtain a second weight vector.

In this step, the possibility of incurring of the state of illness corresponding to the case-history datum by each of the candidate diseases may be calculated based on the common knowledge relevant to the symptoms and the diseases in the case-history datum in the predetermined knowledge graph, to obtain at least two second possibility weights. Moreover, regarding the preset diseases other than the candidate diseases in the preset-disease set, the second possibility weight with which the case-history datum is generated due to those preset diseases may be determined to be 0. Subsequently, the second possibility weights are converted into the required vector forms, to obtain the second weight vector.

Step 105: fusing the first weight vector and the second weight vector, to obtain the disease-analysis vector corresponding to the case-history datum.

In an embodiment of the present disclosure, the first weight vector refers to the possibility of incurring of the state of illness corresponding to the case-history datum by each of the preset diseases that is obtained based on the semantic analysis on the case-history datum, and the second weight vector refers to the possibility of incurring of the state of illness corresponding to the case-history datum by each of the preset diseases that is obtained based on the common knowledge in the knowledge graph. In other words, the first weight vector is the analysis result obtained based on the individual situation, the second weight vector is the analysis result obtained based on common situations, and they are analysis results of the same one case-history datum from different aspects.

In this step, the first weight vector and the second weight vector obtained by the analysis on the case-history datum based on the different aspects may be fused, so as to, by referring to the individual situation and the common situations, obtain a comprehensive result of the analysis on the case-history datum. i.e., the disease-analysis vector corresponding to the case-history datum. The disease-analysis vector does not only take into consideration the individual differences, but also takes into consideration the universal situations. Therefore, the disease-analysis vector can comprehensively reflect the case-history datum from the two aspects, the individual situation and the common situations, thereby realizing the comprehensive analysis on the case-history datum, so as to obtain an all-around analysis result. Therefore, the method for processing medical data according to the embodiments of the present disclosure can enhance the semantic representation of the case-history datum by using the external knowledge of the knowledge graph, and can satisfy more comprehensive demands on analysis on medical data.

In the embodiments of the present disclosure, on one hand, the possibility of incurring of the state of illness corresponding to the case-history datum by each of the preset diseases can be obtaining by semantic analysis on the case-history datum, to generate the first weight vector. On the other hand, the possibility of incurring of the state of illness corresponding to the case-history datum by each of the preset diseases can be obtaining by analyzing the case-history datum by using the common knowledge in the predetermined knowledge graph, to generate the second weight vector. Subsequently, the first weight vector obtained based on the individual situation and the second weight vector obtained based on the common situations can be fused, to obtain the disease-analysis vector corresponding to the case-history datum. The disease-analysis vector can comprehensively reflect one case-history datum from the two aspects, the individual situation and the common situations, thereby enhancing the semantic representation of the case-history datum by using the external knowledge of the knowledge graph, which realizes more comprehensive analysis on the case-history datum.

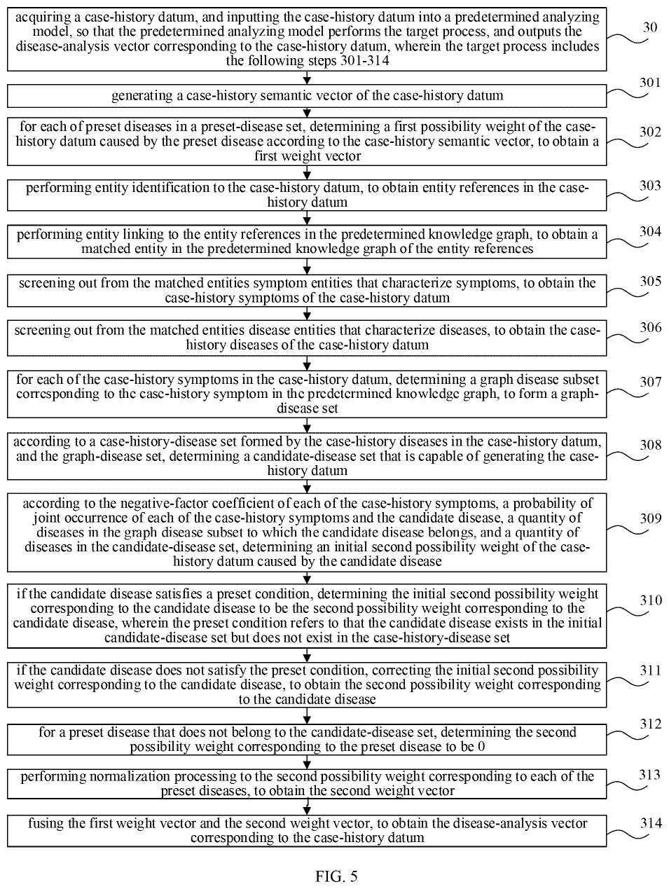

FIG. 4 shows a flow chart of the process of training a model for implementing a method for processing medical data according to an embodiment of the present disclosure. FIG. 5 shows a flow chart of the process of using a model for implementing a method for processing medical data according to an embodiment of the present disclosure. The method can obtain the disease-analysis vector corresponding to one case-history datum by performing the target process. The target process may be implemented by a predetermined analyzing model. Optionally, the predetermined analyzing model may include multiple sub-models having different functions, for example, a semantic-analysis model and a nonlinear-fusion model, and, correspondingly, the method may include the model training process shown in FIG. 4 and the model using process shown in FIG. 5.

Firstly, the model training process:

Step 201: acquiring a case-history-datum training set and a case-history-datum test set.

This step may include firstly using the mode of acquiring the case-history datum in the step 10, to obtain a labelless case-history datum, and subsequently adding a standardized analysis label to the obtained case-history datum, thereby obtaining a labeled case-history datum. The analysis label is the known disease-analysis vector corresponding to the case-history datum, and the type of the analysis label is the type of the preset diseases in the preset-disease set. Subsequently, part of the labeled case-history datum is used as the training datum, to form the case-history-datum training set, and the remaining labeled case-history datum is used as the testing datum, to form the case-history-datum test set.

Step 202: according to the case-history-datum training set and a predetermined loss function, training an original analyzing model, to obtain an intermediate analyzing model.

The loss function may be used to assess the degree of the difference between the predicted value and the true value of the model, and the model may be trained and assessed by minimizing the loss function.

In this step, after each time one training datum having an analysis label (i.e., the true value) has been inputted into an original analyzing model to cause the original analyzing model to output one result (i.e., the predicted value), this step may include, according to the true value and the predicted value corresponding to the training datum, calculating the function value of the predetermined loss function, performing parameter adjustment to the model according to the function value, after the adjustment inputting a training datum into the model again, and repeating the process, till all of the training data in the case-history-datum training set have been completely inputted, to obtain the intermediate analyzing model obtained after the multiple times of parameter adjustment.

Particularly, in an alternative embodiment, the predetermined loss function L may be as

$$L = - \frac{1}{N} {\sum }_{i = 1}^{N} [ {y}_{i} log {P}_{i} + ( 1 - {y}_{i} ) log ( 1 - {P}_{i} ) ]$$

wherein N is the quantity of the types of the analysis labels, i.e., the quantity of the preset diseases in the preset-disease set, yᵢ∈{0,1}, and yᵢ is the true value of the training datum generated by the i-th type of the preset diseases. For example, if the analysis label indicates that a certain training datum is generated by the first type of the preset diseases (assuming that there are totally 8 preset disease and N=8), then y₁ is 1, and all of y₂-y₈ are 0. Pᵢ is the predicted value of the training datum obtained by using the model, and its magnitude represents the possibility with which the training datum is generated by the i-th type of the preset diseases.

Step 203: according to the case-history-datum test set, testing the intermediate analyzing model, to obtain the predetermined analyzing model.

In this step, each of the testing data in the case-history-datum test set may be sequentially inputted into the intermediate analyzing model obtained by the training, thereby, by comparing to obtain the difference between the true value and the predicted value of the testing data, testing the intermediate analyzing model. If the intermediate analyzing model can also effectively predict with respect to the case-history-datum test set, then the test passes, and the intermediate analyzing model is deployed into an electronic device, to obtain the predetermined analyzing model. If the intermediate analyzing model has a poor effect of prediction with respect to the case-history-datum test set, then the test does not pass, subsequently the intermediate analyzing model continues to undergo training and parameter adjustment, till the test passes, and subsequently the model that has passed the test is deployed into an electronic device, to obtain the predetermined analyzing model.

In practical applications, whether the intermediate analyzing model can also effectively predict with respect to the case-history-datum test set may be determined by using various existing model evaluating indicators, for example, the Area Under Curve (referred to for short as the AUC value) of a Receiver Operating Characteristic Curve (referred to for short as a ROC curve), and so on, which is not limited by the embodiments of the present disclosure.

Secondly, the model using process:

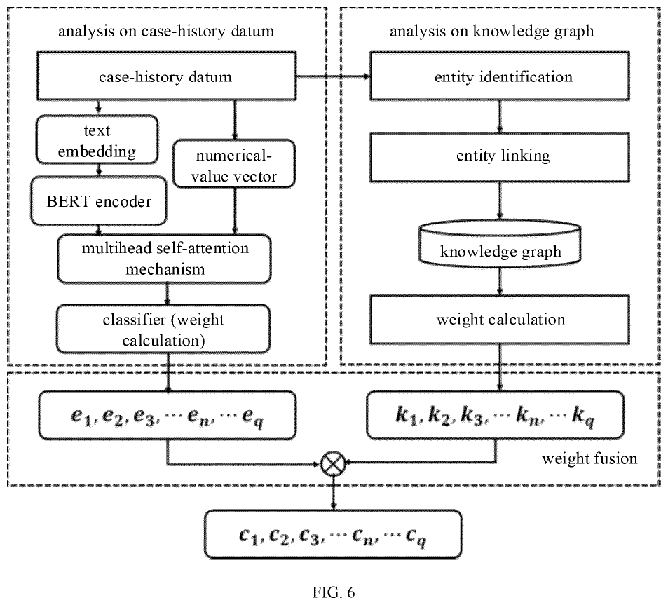

The model using process will be described particularly below with reference to the architecture of the predetermined analyzing model shown in FIG. 6.

Step 30: acquiring a case-history datum, and inputting the case-history datum into a predetermined analyzing model, whereby the predetermined analyzing model performs the target process, and outputs the disease-analysis vector corresponding to the case-history datum, wherein the target process includes the following steps 301-314.

The particular implementation of acquiring the case-history datum may refer to the step 10, and is not discussed further herein.

Step 301: generating a case-history semantic vector of the case-history datum.

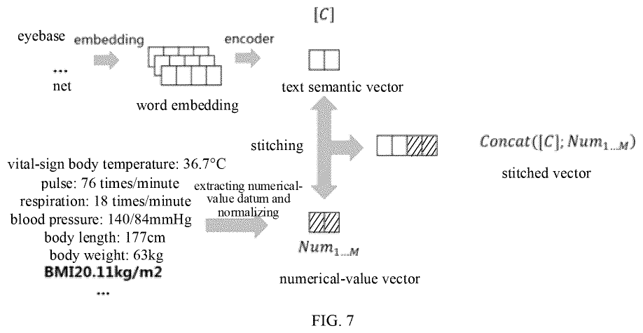

In particular applications, the case-history datum usually includes a text datum such as the past medical history and a numerical-value datum such as an inspection result (for example, the blood-sugar value, the blood-pressure value and so on), and both of the text datum and the numerical-value datum are very important to the analysis on the case-history datum. Therefore, referring to FIG. 7, in this step, the case-history semantic vector of the case-history datum may be generated particularly by using the following steps S11-S14:

S11: encoding the text datum into a text semantic vector.

Optionally, a Bidirectional Encoder Representation from Transformers (BERT) model may be used as the encoder to encode the text datum, to obtain the text semantic vector.

Particularly, the BERT model may firstly convert the text datum into a vector representation, and initialize to obtain an inputted vector. The inputted vector includes: firstly, a word vector (also referred to as a word embedding), which is the vector representation of the words in the text, marks the start of the text by using the mark [cis], and marks the end of a sentence by using the mark [sep]; secondly, a sentence vector (also referred to as a sentence embedding), which is used to distinguish different sentences; and thirdly, a position vector (also referred to as a position embedding), which is used to cause the BERT model to learn the sequential attribute of the text. Subsequently, the BERT model may, by using the self-attention mechanism therein and a feedforward network, encode the inputted vector, and output the text semantic vector. The text semantic vector can describe the semantic representation of the text in the case history.

S12: converting the numerical-value datum into a vector, to obtain a numerical-value vector.

Moreover, the numerical-value datum in the case-history datum may be directly converted into the vector form, wherein the meaning represented by each of the numerical values in the vector is prescribed; for example, the first numerical value in the vector represents the blood-sugar value, the second numerical value in the vector represents the blood-pressure value, and so on. Subsequently, the vector may undergo a softmax normalization processing, thereby obtaining the numerical-value vector. The normalization processing causes each of the components of the numerical-value vector to be limited within a certain interval, for example, [0,1], wherein the numerical-value vector refers to the normalized vector of the numerical-value datum.

S13: stitching the text semantic vector and the numerical-value vector, to obtain a stitched vector.

For example, the text semantic vector particularly includes n vectors, and each of the vectors has a components. The numerical-value vector particularly includes m vectors, and each of the vectors also has a components. The numerical-value vector may be stitched subsequent to the text semantic vector, and the obtained stitched vector includes (n+m) vectors, wherein the last one vector is the numerical-value vector.

If the number of the numerical-value data in the case-history datum are less than a, the other positions of the numerical-value vector may be filled with 0, to reach a numerical values.

S14: by using a multihead self-attention mechanism, encoding the stitched vector, to obtain the case-history semantic vector of the case-history datum.

Subsequently, the stitched vector may be encoded by using a multihead self-attention mechanism, thereby fusing the numerical-value information and the text information in the case-history datum. That can enhance the semantic representation of the case-history datum by using the numerical-value information in the case-history datum, and can satisfy further demands on data analysis.

Particularly, the multihead self-attention mechanism may be expressed as the following formulas:

$$X = Concat ( [ C ] ; {Num}_{1 … M} ) \\ ( {XW}_{i}^{Q} , {XW}_{i}^{K} , {XW}_{i}^{V} ) = ( {Q}_{i} , {K}_{i} , {V}_{i} ) \\ {head}_{i} = Attention ( {Q}_{i} , {K}_{i} , {V}_{i} ) = softmax ( \frac{{Q}_{i} {K}_{i}^{T}}{\sqrt{{d}_{K}}} ) {V}_{i} \\ [ {C}^{′} ] = Concat [ ( {head}_{1} , … , {head}_{t} , … , {head}_{h} ) {W}^{0} ]$$

wherein Concat([C];Num_{1 . . . M}) is the stitched vector of the text semantic vector [C] and the numerical-value vector Num_{1 . . . M}, head, is the i-th head of the totally h heads of the multihead self-attention mechanism, [C′] is the obtained case-history semantic vector, and Wₜ^{Q}, Wᵢ^{K}, Wₜ^{V}, W^{O} are trainable parameters. In an alternative embodiment, h=8, and d_{K}=64.

The essence of the attention mechanism is a mapping from an inquiring statement query (i.e., Q in the formulas) to the key-value pair (i.e., K and V in the formulas) of a target statement, which, by allocating the limited attention to the different characteristic vectors, quickly screens out the critical information having a large contribution to assisting in the diagnosis. Because the case-history datum has a large quantity of long sequences, it easily has the problem of semantic attenuation, and the self-attention mechanism can directly calculate the dependency relation between two characters at any positions, to break through the restriction by the lengths of the sentences. Therefore, when the attention score is being calculated, the same self-attentions of the inquiring statement and the target statement are considered. However, the multihead self-attention mechanism is based on the self-attention mechanism, and is the result of multiple times of self-attention calculation, which enables the BERT model to learn the semantic characteristics from different semantic spaces.

In addition, the number information in the case-history datum also has a very strong decisive effect on the data analysis. Therefore, when the inquiring statement and the target statement are being constructed, the text semantic vector and the numerical-value vector are stitched, and, subsequently, by using the multithread attention mechanism, the dependency relations between the texts and the checking numerical values that have a high distance therebetween in the expression of the case-history text can be extracted excellently, which facilitates the deep analysis on the case-history datum.

In another alternative embodiment of the steps S13-S14, the potential association between the text semantic vector and the numerical-value vector may be found out by using a mutual-attention mechanism. It should be noted that the self-attention mechanism includes firstly stitching the text semantic vector and the numerical-value vector, and subsequently multiplying the stitched vector by Q and K, while the mutual-attention mechanism may include multiplying the text semantic vector by Q, and multiplying the numerical-value vector by K, and does not require the vector stitching.

Step 302: for each of preset diseases in a preset-disease set, determining a first possibility weight of the case-history datum caused by the preset disease according to the case-history semantic vector, to obtain a first weight vector.

In this step, the first weight vector may be obtained by using a classifier. Particularly, the case-history semantic vector obtained by the fusion by using the multihead self-attention mechanism may be inputted into a softmax classifier, wherein the classification categories provided in the softmax classifier are the preset diseases, whereby the softmax classifier can output the first weight vector corresponding to the case-history datum, wherein the components in the first weight vector are the first possibility weights.

Step 303: performing entity identification to the case-history datum, to obtain entity references in the case-history datum.

In an alternative embodiment, this step 303 may particularly include:

S21: according to a predetermined dictionary containing a plurality of entity names, performing entity identification to the case-history datum, to obtain the entity references in the case-history datum.

The entity identification on the case-history datum may particularly employ an entity-identification method based on a dictionary. The languages for describing the same one thing in the case-history datum are not necessarily standard; for example, all of the standard terminology, the vulgo, the shortened form, and so on, of a thing refer to the same one thing, and may be referred to as entity references. The predetermined dictionary may contain the standard terminology, the vulgo, the shortened form, and so on, of multiple particular things, whereby, by using the predetermined dictionary, the entities in the case-history datum can be identified out.

In an embodiment of the present disclosure, according to the demands on the data analysis, the predetermined dictionary may be the Chinese Symptom Knowledge Graph, the disease set in the International Classification of Diseases (ICD-10), the disease symptom entity set in the diabetes knowledge graph issued jointly by Alibaba and the Ruijin Hospital. and so on, which is not limited in the present disclosure.

Further optionally, the step S21 may particularly include:

according to the predetermined dictionary containing the plurality of entity names, performing entity identification to the case-history datum by using a bidirectional maximum matching algorithm, to obtain the entity references in the case-history datum.

The medical data have higher specialty than that of daily expressions, and therefore the entity identification on the medical data is more difficult. The bidirectional maximum-matching method can compare the results of word segmentation obtained by the forward-direction maximum-matching method and the reverse-direction maximum-matching method, thereby determining the correct word-segmentation method, so as to match many medicine-relevant entities to the largest extent, which facilitates the analysis on the medical data.

Step 304: performing entity linking to the entity references in the predetermined knowledge graph, to obtain matched entities in the predetermined knowledge graph of the entity references.

This step can find out which entities the entity references in the case-history datum correspond to in the predetermined knowledge graph.

Optionally, this step 304 may particularly include the following steps S31-S32:

S31: for each of the entities included by the predetermined knowledge graph, calculating similarities between the entity references and each of the entities.

S32: linking the entity references to a target entity corresponding to a largest similarity of the similarities, to use the target entity as the matched entity in the predetermined knowledge graph of the entity references.

In an embodiment of the present disclosure, entity linking may be performed based on the similarities between the entity references in the case-history datum and the entities in the predetermined knowledge graph. Particularly, the similarities between the entity references and each of the entities in the predetermined knowledge graph may be calculated, and the graph entity corresponding to the maximum similarity value may be considered as the entity indicated by that entity reference.

In an alternative embodiment, the step S31 includes:

S311: for any one of the entities, by using at least two similarity calculating modes, calculating initial similarities between the entity references and the entity.

S312: calculating an average value of the initial similarities that are obtained by the calculation, to obtain a similarity between the entity references and the entity.

The similarities between the entity references and the entities in the graph may be calculated in multiple manners, and subsequently the average value of the similarities is solved, which improves the reliability of the result of the entity linking.

In particular applications, optionally, the initial similarities may include at least two of an edit-distance similarity, a Jaccard similarity, a longest-common-substring similarity, a cosine similarity, an explicit-semantic-analysis similarity and a deep-learning similarity.

For example, regarding any one of the entity references in the case-history datum and any one of the entities in the predetermined knowledge graph, the edit-distance similarity Sim_{id}, the Jaccard similarity Sim_{jacc} and the longest-common-substring similarity Sim_{lcs} between the entity reference and the entity may be calculated by using the following formulas.

$${Sim}_{ld} = \frac{{levE}_{R} , {E}_{{K}_{i}} ( \vert {E}_{R} \vert , \vert {E}_{{K}_{i}} \vert )}{max ( \vert {E}_{R} \vert , \vert {E}_{{K}_{i}} \vert )}$$

$${Sim}_{jacc} = jaccard ( bigram ( \vert {E}_{R} \vert ) , bigram ( \vert {E}_{{K}_{i}} \vert ) ) = \frac{\vert bigram ( \vert {E}_{R} \vert ) ⋂ bigram ( \vert {E}_{{K}_{i}} \vert ) \vert}{\vert bigram ( \vert {E}_{R} \vert ) ⋃ bigram ( \vert {E}_{{K}_{i}} \vert ) \vert}$$

$${Sim}_{lcs} = \frac{\vert lcs ( {E}_{R} , {E}_{{K}_{i}} ) \vert}{max ( \vert {E}_{R} \vert , \vert {E}_{{K}_{i}} \vert )}$$

In the above formulas, levE_{R}, E_{K}_{ᵢ} (|E_{R}|,|E_{K}_{ᵢ}|) represents at least how many times of processing (including deletion, addition and substitution) are required to change the entity reference E_{R} into an entity E_{K}_{ᵢ} in the predetermined knowledge graph; max(|E_{R}|, |E_{K}_{ᵢ}|) represents the maximum length of E_{R} and E_{K}_{ᵢ}; bigram(|E_{R}|) represents a binary group obtained after binary word-segmentation of E_{R}; bigram(|E_{K}_{ᵢ}|) represents a binary group obtained after binary word-segmentation of the entity reference E_{K}_{ᵢ}; and |lcs(E_{R},E_{K}_{ᵢ})| represents the maximum length of the same substrings of E_{R} and E_{K}_{ᵢ}.

The similarity between the entity reference and the entity may be obtained by calculation by using the following formula:

Sim(E_{R},E_{K}_{ᵢ})=(Sim_{id}+Sim_{jacc}+Sim_{lcs})/3

In an embodiment of the present disclosure, the calculation of the similarities between the case-history entities and the graph entities comprehensively takes into consideration the results of multiple similarities such as the edit-distance similarity, the Jaccard similarity and the longest-common-substring similarity, so as to measure the degrees of the similarities between the case-history entities and the graph entities from different perspectives, and subsequently solve the average value of the multiple similarities obtained from the different perspectives, which can enable the result of the entity linking to be more reliable.

Step 305: screening out from the matched entities symptom entities that characterize symptoms, to obtain the case-history symptoms of the case-history datum.

In an embodiment of the present disclosure, a list of various types of entities is constructed in the predetermined knowledge graph. In this step, the entities that exist in the symptom-entity list in the predetermined knowledge graph may be screened from the matched entities obtained in the step 304, thereby obtaining m symptoms that are referred to in the case-history datum (for example, referred to in the self-reporting), which are referred to as the case-history symptoms S_{Ri}, to form the following case-history-symptom set S_{R}.

S_{R}={S_{R}_{₁},S_{R}_{₂}, . . . ,S_{R}_{ᵢ}, . . . ,S_{R}_{ₘ}}

Step 306: screening out from the matched entities disease entities that characterize diseases, to obtain the case-history diseases of the case-history datum.

As similar to the step 305, in this step, the entities that exist in the disease-entity list in the predetermined knowledge graph may be screened from the matched entities obtained in the step 304, thereby obtaining p diseases that are referred to in the case-history datum (for example, referred to in the primary-diagnosis result), which are referred to as the case-history diseases d_{Ri}, to form the following case-history-disease set Da.

D_{R}={(d_{R}_{₁}:f_{R}_{₁}),(d_{R}_{₂}:f_{R}_{₂}), . . . ,(d_{R}_{ᵢ}:f_{R}_{ᵢ}), . . . ,(d_{R}_{ₚ}:f_{R}_{ₚ})}

wherein f_{Ri} is the probability of occurrence of the case-history disease d_{Ri} in the predetermined knowledge graph, and represents the magnitude of the possibility with which the case-history disease d_{Ri} happens in life. The probability value of the probability of occurrence may be stored in the predetermined knowledge graph as an attribute of the case-history disease d_{Ri}, and may also be stored in the predetermined knowledge graph as a tail entity corresponding to the case-history disease d_{Ri}.

Step 307: for each of the case-history symptoms in the case-history datum, determining a graph disease subset corresponding to the case-history symptom in the predetermined knowledge graph, to form a graph-disease set.

In this step, the case-history symptom may be used as the head entity, and the disease tail entity is matched in the predetermined knowledge graph, thereby obtaining the graph disease corresponding to the case-history symptom in the predetermined knowledge graph.

According to experience, one symptom may be the indication of more than one disease. Therefore, one case-history symptom S_{Ri} might match with n graph diseases in the predetermined knowledge graph, and the n graph diseases form the graph disease subset Dₜ={dₜ₁, dₜ₂, . . . , dₜⱼ, . . . , dₜₙ} corresponding to the case-history symptom S_{Ri}. The graph disease subsets corresponding to all of the case-history symptoms in one case-history datum form the graph-disease set D.

Step 308: according to a case-history-disease set formed by the case-history diseases in the case-history datum, and the graph-disease set, determining a candidate-disease set that might generate the case-history datum.

This step 308 may be executed with the following steps S41-S43:

S41: for each of the case-history diseases in the case-history-disease set, if a negative-factor coefficient of the case-history disease is a preset minimum value, deleting the case-history disease from the case-history-disease set, wherein the case-history-disease set obtained after the case-history disease is deleted forms a target-case-history-disease set.

The negative-factor coefficient f_{negD} of the case-history disease is used to define the negative-direction influence on the case-history disease by a negating word, and the negative-factor coefficient f_{negD} of the case-history disease represents the degree of semantic negation to the case-history disease in the case-history description. If the negative-factor coefficient f_{negD} of a certain case-history disease is the preset minimum value, that represents that the case-history disease is completely negated in the case-history description. For example, if in the past medical history of the case history it is described that “hepatitis history is denied”, then the negative-factor coefficient f_{negD} of the case-history disease “hepatitis” is set to be the preset minimum value, for example, −1.

Therefore, if the negative-factor coefficient f_{negD} of a certain case-history disease is the preset minimum value, then the case-history disease may be deleted from the case-history-disease set D_{R}. By determining the values of the negative-factor coefficients of all of the case-history diseases in the case-history-disease set D_{R}, the case-history diseases that are clearly negated can be excluded from the case-history-disease set, thereby obtaining the target-case-history-disease set D_{R}′. Accordingly, all of the diseases in the target-case-history-disease set D_{R}′ are diseases having possibility in the perspective of the case-history description, and can be considered as the diseases that might cause the state of illness corresponding to the case-history datum.

Additionally, how to decide the value of the negative-factor coefficient f_{negD} of the case-history disease will be described below.

S42: for each of the case-history diseases in the case-history-disease set, if the negative-factor coefficient of the case-history disease is the preset minimum value, and the case-history disease exists in the graph-disease set, deleting the case-history disease from the graph-disease set, wherein the graph-disease set obtained after the case-history disease is deleted forms an initial candidate-disease set.

Regarding the case-history disease that is clearly negated in the description of the case-history datum, because the negating expression thereon in the preceding context of the case history influences the attribute of the case-history disease, and reversely excludes the possibility with which the case-history datum is caused by the case-history disease, it is further required to exclude it from the graph-disease set D; in other words, the second possibility weight corresponding to the case-history disease should be 0, thereby obtaining the initial candidate-disease set D′. Accordingly, all of the diseases in the initial candidate-disease set D′ are diseases having possibility in the perspective of the graph-knowledge description, and can be considered as the diseases that might cause the state of illness corresponding to the case-history datum.

S43: for each of target case-history diseases included by the target-case-history-disease set, if the target case-history disease does not exist in the initial candidate-disease set, adding the target case-history disease into the initial candidate-disease set, to form the candidate-disease set that might generate the case-history datum.

Subsequently, regarding each of the diseases in the target-case-history-disease set, if the disease does not exist in the initial candidate-disease set D′, that indicates that although the disease does not have possibility from the perspective of the graph knowledge, it still has possibility from the perspective of the case-history description. Therefore, the disease may be added into the initial candidate-disease set D′, to obtain all of the candidate diseases that might cause the state of illness corresponding to the case-history datum from both of the two perspectives of the graph knowledge and the case-history description, thereby forming the candidate-disease set D″.

By using the steps S41-S43, all of the candidate diseases that might cause the state of illness corresponding to a certain case-history datum from both of the two perspectives of the graph knowledge and the case-history description can be screened out, which cannot only prevent omitting a possible disease, thereby increasing the accuracy of the analysis result, but also can prevent unnecessary data processing on the diseases that are clearly negated in the case-history description, thereby increasing the efficiency of the data processing.

Step 309: according to the negative-factor coefficient of each of the case-history symptoms, a probability of joint occurrence of each of the case-history symptoms and the candidate disease, a quantity of diseases in the graph disease subset that the candidate disease belongs to, and a quantity of diseases in the candidate-disease set, determining an initial second possibility weight of the case-history datum caused by the candidate disease.

In an alternative embodiment, regarding each of the candidate diseases dᵢⱼ in the candidate-disease set D″, according to the negative-factor coefficient f_{negS} of each of the case-history symptoms S_{Ri}, the probability of joint occurrence p(S_{Ri}, dᵢⱼ) of each of the case-history symptoms S_{Ri} and the candidate disease dᵢⱼ, the quantity |Dᵢ| of the diseases in the graph disease subset Di that the candidate disease dᵢⱼ belongs to, and the quantity |D″| of the diseases in the candidate-disease set D″, the initial second possibility weight Wdᵢⱼ of incurring of the case-history datum by the candidate disease do may be determined by using the following formula.

$${W}_{{d}_{ij}} = {\sum }_{{S}_{{R}_{s}} ∈ {S}_{R}} \frac{{f}_{negS} \times p ( {s}_{{R}_{i}} , {d}_{ij} )}{{\sum }_{qr ∈ {Q}_{ij}} p ( {q}_{r} , {d}_{ij} )} {log}_{2} \frac{\vert D ″ \vert}{\vert {D}_{i} \vert + 1}$$

Regarding each of the candidate diseases dᵢⱼ in the candidate-disease set D″, the predetermined knowledge graph has one symptom set S_{d}_{ᵢⱼ}{=S_{d}_{ᵢⱼ}₁, S_{d}_{ᵢⱼ}₂, . . . , S_{d}_{ᵢⱼ}ᵢ, . . . , S_{d}_{ᵢⱼ}_{M}} corresponding thereto, which includes M symptoms relevant to the disease in the predetermined knowledge graph, wherein in the above formula, Qᵢⱼ=S_{R}∩S_{d}_{ᵢⱼ}.

The negative-factor coefficient f_{negS} of the case-history symptom is used to define the is negative-direction influence on the case-history symptom by a negating word, and the negative-factor coefficient f_{negS} of the case-history symptom represents the degree of semantic negation to the case-history symptom in the case-history description. If the negative-factor coefficient f_{negS} of a certain case-history symptom is the preset minimum value, that represents that the case-history symptom is completely negated in the case-history description. For example, if in the body-inspection result of the case history “no hand-foot tremor” is described, then the negative-factor coefficient f_{negS} of the case-history symptom “hand-foot tremor” is set to be the preset minimum value, for example, −1.

The probability of joint occurrence p(S_{Ri}, dᵢⱼ) of the case-history symptom S_{Ri} and the candidate disease dᵢⱼ may be acquired from the predetermined knowledge graph. The probability value of the probability of joint occurrence may be stored in the predetermined knowledge graph as an attribute of the case-history symptom S_{Ri} or the case-history disease d_{Ri}, and may also be stored in the predetermined knowledge graph as a tail entity corresponding to the case-history symptom S_{Ri} or the case-history disease d_{Ri}.

Correspondingly, before the step of determining the second possibility weight, the method may further include the following steps: acquiring from the predetermined knowledge graph a probability of joint occurrence of each of the case-history symptoms and the candidate disease.

It can be seen from the above formula that, if the quantity (i.e., |D₁) of the graph diseases corresponding to the case-history symptom S_{Ri} is lower,

$${log}_{2} \frac{\vert {D}^{″} \vert}{\vert {D}_{i} \vert + 1}$$

is higher. When f_{negS} is greater than 0, if the probability of joint occurrence of the case-history symptom S_{Ri} and the candidate disease dᵢⱼ is higher

$$\frac{{f}_{negS} \times p ( {s}_{{R}_{i}} , {d}_{ij} )}{{\sum }_{qr ∈ {Q}_{ij}} p ( {q}_{r} , {d}_{ij} )}$$

is higher. When f_{negS} is less than 0, if the probability of joint occurrence of the case-history symptom SL and the candidate disease dᵢⱼ is higher,

$$\frac{{f}_{negS} \times p ( {S}_{{R}_{i}} , {d}_{ij} )}{{\sum }_{qr ∈ {Q}_{ij}} p ( {q}_{r} , {d}_{ij} )}$$

is lower.

Additionally, how to decide the value of the negative-factor coefficient f_{negS} of the case-history symptom will be described below.

Step 310: if the candidate disease satisfies a preset condition, determining the initial second possibility weight corresponding to the candidate disease to be the second possibility weight corresponding to the candidate disease, wherein the preset condition refers to that the candidate disease exists in the initial candidate-disease set but does not exist in the case-history-disease set.

It can be known according to the steps S41-S42 that the candidate diseases in the candidate-disease set may come from the case-history-disease set, i.e., come from the case-history description, and may also come from the graph-disease set, i.e., come from the graph knowledge. Moreover, the source of the candidate disease indicates whether the candidate disease places emphasis on the gain from the individual situation, or places emphasis on the gain from the universal knowledge. Therefore, in an embodiment of the present disclosure, it is required to readjust the initial second possibility weights of the candidate diseases by referring to the different sources of the candidate diseases.

Particularly, if a candidate disease comes from the gain from the graph knowledge, but is not referred to in the case-history description, then it may be considered that the candidate disease might be a disease that is determined based on the universal situations. In this case, the initial second possibility weight corresponding to the candidate disease may be directly determined to be the second possibility weight corresponding to the candidate disease; in other words, the case-history datum is analyzed with respect to the second possibility weight directly according to the graph knowledge.

Moreover, in the case in which the candidate disease also exists in the case-history description (including the case in which the candidate disease exists in both of the graph-disease set and the case-history-disease set, and the case in which the candidate disease exists in merely the case-history-disease set), that indicates that the case-history description has already had a clear analysis result with respect to the individual situation. Accordingly, it is required to, by referring to the individual situation, correct the initial second possibility weight corresponding to the candidate disease, which particularly includes the following step 311.

Step 311: if the candidate disease does not satisfy the preset condition, correcting the initial second possibility weight corresponding to the candidate disease, to obtain the second possibility weight corresponding to the candidate disease.

This step 311 may particularly include the following steps S51-S52:

S51: if the candidate disease exists in both of the case-history-disease set D_{R} and the initial candidate-disease set D′ (the candidate disease belongs to D_{R}, with the identifier of d_{Ri}), according to the probability of occurrence f_{Ri} of each of the candidate diseases, correcting the initial second possibility weight Wd_{Ri} corresponding to the candidate disease d_{Ri}, to obtain the second possibility weight W′d_{Ri} corresponding to the candidate disease d_{Ri}.

In the step S51, the second possibility weight W′dᵢⱼ may be obtained by calculation by using the following formula:

$${W}_{{d}_{{R}_{i}}}^{′} = {W}_{{d}_{{R}_{i}}} ( 1 + \frac{{f}_{{R}_{i}}}{{\sum }_{{f}_{{R}_{i}} ∈ {D}_{R}} {f}_{{R}_{i}}} )$$

wherein Wd_{Ri} refers to the initial second possibility weight corresponding to the candidate disease d_{Ri} in the initial second possibility weights.

The above formula indicates that the degree of contribution on the analysis result by the disease clarified in the case-history description is greater than the degree of contribution on the analysis result by the symptom described in the case history.

Because the above formula uses the probability of occurrence f_{Ri} of each of the candidate diseases, correspondingly, before the step of correcting the initial second possibility weight corresponding to the candidate disease, the method further includes: acquiring from the predetermined knowledge graph a probability of occurrence of each of the candidate diseases.

S52: if the candidate disease exists in the case-history-disease set D_{R} but does not exist in the initial candidate-disease set D′ (the candidate disease belongs to D_{R}, with the identifier of d_{Ri}), according to the negative-factor coefficient f_{negD} of the candidate disease d_{Ri}, a preset hyper-parameter β and the quantity |D″| of the diseases in the candidate-disease set D″, correcting the initial second possibility weight Wdᵢ corresponding to the candidate disease d_{Ri}, to obtain the second possibility weight W′d_{Ri} corresponding to the candidate disease d_{Ri}.

In the step S52, the second possibility weight W′dᵢⱼ may be obtained by calculation by using the following formula:

$${W}_{{d}_{{R}_{i}}}^{′} = {f}_{negD} \times \frac{β}{\vert {D}^{″} \vert} {\sum }_{{d}_{i} ∈ {D}^{″}} {W}_{{d}_{i}}$$

wherein Wdᵢ refers to the initial second possibility weight corresponding to the candidate disease di in the initial second possibility weights. β is a preset hyper-parameter, and β≥1. In an alternative embodiment, it may be preset that β=1.5.

The above formula indicates that the degree of contribution on the analysis result by the disease clarified in the case-history description is greater than the degree of contribution on the analysis result by the symptom described in the case history.

Additionally, before determining the value of the candidate disease, the method may further include, by using the following steps S61-S62, determining the value of the negative-factor coefficient f_{negD} of each of the case-history diseases d_{Ri}, and the value of the negative-factor coefficient f_{negS} of each of the case-history symptoms S_{Ri}.

S61: according to a degree of negation to the case-history symptom by a first neighboring word located at a position preceding the case-history symptom in the case-history datum, determining the negative-factor coefficient of the case-history symptom, wherein the negative-factor coefficient of the case-history symptom is negatively correlated with the degree of negation to the case-history symptom by the first neighboring word.

If the preceding context of the case-history symptom totally changes the semantic information, then the negative-factor coefficient f_{negS} of the case-history symptom may be set to be lower. If the preceding context of the case-history symptom partially changes the semantic information, then the negative-factor coefficient f_{negS} of the case-history symptom may be set to be higher.

For example, in the case in which the preceding context of the case-history symptom such as “no hand-foot tremor” is the first type of negating words such as “no” and “not”, the word “no” totally changes the semantics of “hand-foot tremor”, whereby the semantics changes from affirming “hand-foot tremor” into negating “hand-foot tremor”. Therefore, the negative-factor coefficient f_{negS} of the case-history symptom “hand-foot tremor” may be set to be −1.

As another example, in the case in which the preceding context of the case-history symptom such as “involuntary hand-foot tremor” is the second type of negating words such as “involuntary”, the word “involuntary” partially changes the semantics of “hand-foot tremor”, is a further limitation on “hand-foot tremor”, and is not negation of “hand-foot tremor”. Therefore, the negative-factor coefficient f_{negS} of the case-history symptom “hand-foot tremor” may be set to be 0.5.

S62: according to a degree of negation to the case-history disease by a second neighboring word located at a position preceding the case-history disease in the case-history datum, determining the negative-factor coefficient of the case-history disease, wherein the negative-factor coefficient of the case-history disease is negatively correlated with the degree of negation to the case-history disease by the second neighboring word.

As similar to the step S61, if the preceding context of the case-history disease totally changes the semantic information, then the negative-factor coefficient f_{negD} of the case-history disease may be set to be lower. If the preceding context of the case-history disease partially changes the semantic information, then the negative-factor coefficient f_{negD} of the case-history disease may be set to be higher.

For example, in the case in which the preceding context of the case-history symptom such as “denied hepatitis history” is the first type of negating words such as “denied”, the word “denied” totally changes the semantics of “hepatitis”, whereby the semantics changes from affirming “hepatitis” into negating “hepatitis”. Therefore, the negative-factor coefficient f_{negD} of the case-history disease “hepatitis” may be set to be −1.

In practical applications, because the expressions in case histories are fixed, the degree of negation with respect to the symptoms or the diseases by the previous-context words may be determined by presetting various types of negating words.

Step 312: for a preset disease that does not belong to the candidate-disease set, determining the second possibility weight corresponding to the preset disease to be 0.

By using the above steps, the second possibility weights corresponding to each of the candidate diseases that belong to the candidate-disease set are obtained. However, the candidate-disease set cannot necessarily completely cover the preset-disease set, and, therefore, the quantity of the candidate diseases might be less than the quantity of the preset diseases. If the quantity of the candidate diseases is less than the quantity of the preset diseases, that indicates that merely the second possibility weights corresponding to some of the preset diseases are determined. For a preset disease that does not belong to the candidate-disease set, the second possibility weight corresponding to the preset disease is directly determined to be 0.

Step 313: performing normalization processing to the second possibility weight corresponding to each of the preset diseases, to obtain the second weight vector.

The second possibility weight corresponding to each of the preset diseases undergoes the softmax normalization processing, thereby obtaining the second possibility weights obtained after the normalization, and the second possibility weights obtained after the normalization are used as the vector components, to form the second weight vector.

Step 314: fusing the first weight vector and the second weight vector, to obtain the disease-analysis vector corresponding to the case-history datum.

The first weight vector and the second weight vector have equal dimensionalities, the dimensionalities are a quantity of diseases in the preset-disease set, and correspondingly, this step 314 particularly includes the following steps S71-S72:

S71: by using different preset importance coefficients, weighting the first possibility weight and the second possibility weight of the equal dimensionalities, to obtain weighted parameters, wherein a preset importance coefficient corresponding to the first possibility weight and a preset importance coefficient corresponding to the second possibility weight are negatively correlated.

In an alternative embodiment, the preset importance coefficient corresponding to the first possibility weight may be yᵢ, and the preset importance coefficient corresponding to the second possibility weights may be (1−yᵢ), thereby realizing the negative correlation therebetween.

If the preset-disease set includes q preset diseases, the first weight vector K and the second weight vector E are as follows:

K=[k₁,k₂, . . . ,kᵢ, . . . ,k_{q}]

E=[e₁,e₂, . . . ,eᵢ, . . . ,e_{q}]

The first possibility weight kᵢ and the second possibility weights eᵢ of the equal dimensionality may undergo linear weighting by using the following formula, to obtain q weighted parameters zₗ-z_{q}.

zₗ=yᵢeᵢ+(1−yᵢ)kᵢ

wherein yᵢ reflects whether the weighted parameter zₗ is influenced more by the case-history datum, or influenced more by the predetermined knowledge graph, yᵢ may be regulated manually as a hyper-parameter, and may also be obtained by learning by using a neural network, which is not limited in the embodiments of the present disclosure.

S72: by using a linear function or a nonlinear function, calculating the weighted parameters, to obtain fused weights, wherein the fused weights form the disease-analysis vector corresponding to the case-history datum, wherein the disease-analysis vector has a dimensionality equal to the dimensionalities of the first weight vector and the second weight vector.

Optionally, the weighted parameters zₗ-z_{q} may be individually calculated by using a nonlinear function a, to obtain the fused weights cₗ-c_{q}, wherein cₗ-c_{q} represent the illness probabilities corresponding to the first preset disease to the q-th preset disease.

$${c}_{i} = σ ( {z}_{i} ) = \frac{1}{1 - exp ( - {z}_{i} )}$$

In an alternative embodiment, the nonlinear function a may be a sigmoid function.

The process described by using the steps S71-S72 may be expressed as the following formula:

$${c}_{i} = σ ( {γ}_{i} {e}_{i} + ( 1 - {γ}_{i} ) {k}_{i} ) = \frac{1}{1 - exp [ - ( {γ}_{i} {e}_{i} + ( 1 - {γ}_{i} ) {k}_{i} ) ]}$$

In the embodiments of the present disclosure, on one hand, the possibility of incurring of the state of illness corresponding to the case-history datum by each of the preset diseases can be obtaining by semantic analysis on the case-history datum, to generate the first weight vector, and the semantic representation of the case-history text is enhanced by using the numerical-value information. On the other hand, the possibility of incurring of the state of illness corresponding to the case-history datum by each of the preset diseases can be obtaining by analyzing the case-history datum by using the common knowledge in the predetermined knowledge graph, to generate the second weight vector. Subsequently, the first weight vector obtained based on the individual situation and the second weight vector obtained based on the common situations can be fused, to obtain the disease-analysis vector corresponding to the case-history datum. The disease-analysis vector can comprehensively reflect one case-history datum from the two aspects, the individual situation and the common situations, thereby enhancing the semantic representation of the case-history datum by using the external knowledge of the knowledge graph, which realizes more comprehensive analysis on the case-history datum.

Some usage scenes of the analysis result obtained by using the method for processing medical data will be described below. It can be understood that the usage scenes of the analysis result are not limited to the examples of the scenes illustrated below. It should be noted that the acquirement and the usage of the case-history datum and the corresponding analysis result shall be known and permitted by the patient himself, and the acquirement and the usage of the case-history datum and the corresponding analysis result shall comply with the laws and regulations of the place of jurisdiction where the solutions are implemented.

### The First Scene: Medical Education

In medicine-relevant education, the analysis result according to the embodiments of the present disclosure, after artificial checking by professionals such as medical teachers and doctors, may be used as cases for diagnostic analysis, thereby providing a large amount of teaching material for the field of medical education.

### The Second Scene: The Construction of an Automation Interrogation Platform

Currently, increasingly more hospitals have provided the service of automated interrogation, wherein the platform may be used by users by means of software, WeChat Mini Program, WeChat Official Account and so on, and provides preliminary diagnosis and advices for the users by using an automated dialogue robot.

The analysis result according to the embodiments of the present disclosure, after artificial checking by professionals such as doctors, may be used as the learning data for the automated dialogue robot, to construct the replying mechanism of the automated dialogue robot. Furthermore, in order to ensure the correctness and the specialty of the medical advices, the medical advices in the reply by the automated dialogue robot may be firstly checked by professionals before presented to the users.

### The Third Scene: Reference of Doctor Interrogation

Because of the differences between individuals, sometimes the universal situations might be not capable of being applied to the individual situation, or extremely complicated symptoms might result in insufficient diagnosis of common diseases. Therefore, the analysis result according to the embodiments of the present disclosure may be used as reference data for the diagnosis by doctors, so as to serve to notice the doctors. If the diagnostic result of a doctor is different excessively from the analysis result, the doctor may be prompted to perform more careful examination and determination.

The present disclosure further provides an apparatus for predicting a diabetes complication, wherein the apparatus includes a processor, a memory and a program stored in the memory and executable in the processor, and the program, when executed by the processor, implements the steps of the method for processing medical data stated above, to obtain the disease-analysis vector corresponding to the case-history datum, wherein the preset-disease set includes one or more diabetes complications, and each of components of the disease-analysis vector represents an illness probability corresponding to each of the diabetes complications.

The preset diseases in the method for processing medical data may be set to be the diabetes complications. Correspondingly, each of the components in the first weight vector represents the possibility of incurring of each of the diabetes complications by the state of illness corresponding to the case-history datum that is obtained based on the semantic analysis on the case-history datum. Each of the components in the second weight vector represents the possibility of incurring of each of the diabetes complications by the state of illness corresponding to the case-history datum that is obtained based on the graph-knowledge analysis on the case-history datum. Each of the components in the disease-analysis vector represents the possibility of each of the diabetes complications that is obtained by fusing the semantic analysis and the graph-knowledge analysis on the case history.

In an alternative embodiment, the apparatus may be configured to directly output the disease-analysis vector.

In another alternative embodiment, the apparatus may be configured to output the name of the diabetes complication corresponding to the component of the maximum numerical value in the disease-analysis vector.

In a third alternative embodiment, the apparatus may be configured to output the name of the diabetes complication corresponding to the component in the disease-analysis vector whose numerical value is the maximum and is greater than a preset threshold.

In a fourth alternative embodiment, the apparatus may be further configured to output both of the names of the component of the maximum numerical value (or the component whose numerical value is the maximum and is greater than a preset threshold) in the disease-analysis vector and of the diabetes complication corresponding to the component.

The outputting mode may be displaying, playing and so on, which is not limited in the embodiments of the present disclosure.

The result outputted by the apparatus may be applied in the scenes described above.

An embodiment of the present disclosure further discloses a non-transitory computer-readable storage medium, wherein an instruction in the storage medium, when executed by a processor of an electronic device, enables the electronic device to implement the method for processing medical data stated above.

The “one embodiment”, “an embodiment” or “one or more embodiments” as used herein means that particular features, structures or characteristics described with reference to an embodiment are included in at least one embodiment of the present disclosure. Moreover, it should be noted that here an example using the wording “in an embodiment” does not necessarily refer to the same one embodiment.

The description provided herein describes many concrete details. However, it can be understood that the embodiments of the present disclosure may be implemented without those concrete details. In some of the embodiments, well-known processes, structures and techniques are not described in detail, so as not to affect the understanding of the description.

In the claims, any reference signs between parentheses should not be construed as limiting the claims. The word “comprise” does not exclude elements or steps that are not listed in the claims. The word “a” or “an” preceding an element does not exclude the existing of a plurality of such elements. The present disclosure may be implemented by means of hardware comprising several different elements and by means of a properly programmed computer. In unit claims that list several devices, some of those devices may be embodied by the same item of hardware. The words first, second, third and so on do not denote any order. Those words may be interpreted as names.

Finally, it should be noted that the above embodiments are merely intended to explain the technical solutions of the present disclosure, and not to limit them. Although the present disclosure is explained in detail with reference to the above embodiments, a person skilled in the art should understand that he can still modify the technical solutions set forth by the above embodiments, or make equivalent substitutions to part of the technical features of them. However, those modifications or substitutions do not make the essence of the corresponding technical solutions depart from the spirit and scope of the technical solutions of the embodiments of the present disclosure.

1. A method for processing medical data, wherein the method comprises:
acquiring a case-history datum, and performing a target process to obtain a disease-analysis vector corresponding to the case-history datum, wherein the target process comprises:
generating a case-history semantic vector of the case-history datum;
for each of preset diseases in a preset-disease set determining a first possibility weight of the case-history datum caused by the preset disease according to the case-history semantic vector, to obtain a first weight vector;
according to case-history symptoms and case-history diseases in the case-history datum, determining from a predetermined knowledge graph a candidate disease that is capable of generating the case-history datum, wherein the predetermined knowledge graph comprises entities and relations that are relevant to the preset disease, and the candidate disease belongs to the preset-disease set;
determining a second possibility weight of the case-history datum caused by the candidate disease, to obtain a second weight vector; and
fusing the first weight vector and the second weight vector, to obtain the disease-analysis vector corresponding to the case-history datum.

2. The method according to claim 1, wherein the case-history datum comprises a text datum and a numerical-value datum, and the step of generating the case-history semantic vector of the case-history datum comprises:
encoding the text datum into a text semantic vector;
converting the numerical-value datum into a vector, to obtain a numerical-value vector;
stitching the text semantic vector and the numerical-value vector, to obtain a stitched vector; and
by using a multihead self-attention mechanism, encoding the stitched vector, to obtain the case-history semantic vector of the case-history datum.

3. The method according to claim 1, wherein the step of, according to the case-history symptoms and the case-history diseases in the case-history datum, determining from the predetermined knowledge graph the candidate disease that is capable of generating the case-history datum comprises:
for each of the case-history symptoms in the case-history datum, determining a graph disease subset corresponding to the case-history symptom in the predetermined knowledge graph, to form a graph-disease set; and
according to a case-history-disease set formed by the case-history diseases in the case-history datum, and the graph-disease set, determining a candidate-disease set that is capable of generating the case-history datum.

4. The method according to claim 3, wherein the step of, according to the case-history-disease set formed by the case-history diseases in the case-history datum, and the graph-disease set, determining the candidate-disease set that is capable of generating the case-history datum comprises:
for each of the case-history diseases in the case-history-disease set, if a negative-factor coefficient of the case-history disease is a preset minimum value, deleting the case-history disease from the case-history-disease set, wherein the case-history-disease set obtained after the case-history disease is deleted forms a target-case-history-disease set;
for each of the case-history diseases in the case-history-disease set, if the negative-factor coefficient of the case-history disease is the preset minimum value, and the case-history disease exists in the graph-disease set, deleting the case-history disease from the graph-disease set, wherein the graph-disease set obtained after the case-history disease is deleted forms an initial candidate-disease set; and
for each of target case-history diseases comprised by the target-case-history-disease set, if the target case-history disease does not exist in the initial candidate-disease set, adding the target case-history disease into the initial candidate-disease set, to form the candidate-disease set that is capable of generating the case-history datum.

5. The method according to claim 4, wherein the step of determining the second possibility weight of the case-history datum caused by the candidate disease, to obtain the second weight vector comprises:
according to the negative-factor coefficient of each of the case-history symptoms, a probability of joint occurrence of each of the case-history symptoms and the candidate disease, a quantity of diseases in the graph disease subset to which the candidate disease belongs, and a quantity of diseases in the candidate-disease set, determining an initial second possibility weight of the case-history datum caused by the candidate disease;
if the candidate disease satisfies a preset condition, determining the initial second possibility weight corresponding to the candidate disease to be the second possibility weight corresponding to the candidate disease, wherein the preset condition refers to that the candidate disease exists in the initial candidate-disease set but does not exist in the case-history-disease set; and
if the candidate disease does not satisfy the preset condition, correcting the initial second possibility weight corresponding to the candidate disease, to obtain the second possibility weight corresponding to the candidate disease.

6. The method according to claim 5, wherein the step of, if the candidate disease does not satisfy the preset condition, correcting the initial second possibility weight corresponding to the candidate disease, to obtain the second possibility weight corresponding to the candidate disease comprises:
if the candidate disease exists in both of the case-history-disease set and the initial candidate-disease set, according to a probability of occurrence of the candidate disease, correcting the initial second possibility weight corresponding to the candidate disease, to obtain the second possibility weight corresponding to the candidate disease; and
if the candidate disease exists in the case-history-disease set but does not exist in the initial candidate-disease set, according to the negative-factor coefficient of the candidate disease, a preset hyper-parameter and the quantity of the diseases in the candidate-disease set, correcting the initial second possibility weight corresponding to the candidate disease, to obtain the second possibility weight corresponding to the candidate disease.

7. The method according to claim 6, wherein before the step of, according to the case-history symptoms and the case-history diseases in the case-history datum, determining from the predetermined knowledge graph the candidate disease that is capable of generating the case-history datum, the method further comprises:
according to a degree of negation to the case-history symptom by a first neighboring word located at a position preceding the case-history symptom in the case-history datum, determining the negative-factor coefficient of the case-history symptom, wherein the negative-factor coefficient of the case-history symptom is negatively correlated with the degree of negation to the case-history symptom by the first neighboring word; and
according to a degree of negation to the case-history disease by a second neighboring word located at a position preceding the case-history disease in the case-history datum, determining the negative-factor coefficient of the case-history disease, wherein the negative-factor coefficient of the case-history disease is negatively correlated with the degree of negation to the case-history disease by the second neighboring word.

8. The method according to claim 5, wherein after the step of, if the candidate disease does not satisfy the preset condition, correcting the initial second possibility weight corresponding to the candidate disease, to obtain the second possibility weight corresponding to the candidate disease, the method further comprises:
for a preset disease that does not belong to the candidate-disease set, determining the second possibility weight corresponding to the preset disease to be 0; and
performing normalization processing to the second possibility weight corresponding to each of the preset diseases, to obtain the second weight vector.

9. The method according to claim 5, wherein before the step of determining the second possibility weight of the case-history datum caused by the candidate disease, to obtain the second weight vector, the method further comprises:
acquiring from the predetermined knowledge graph a probability of joint occurrence of each of the case-history symptoms and the candidate disease.

10. The method according to claim 6, wherein before the step of, if the candidate disease does not satisfy the preset condition, correcting the initial second possibility weight corresponding to the candidate disease, to obtain the second possibility weight corresponding to the candidate disease, the method further comprises:
acquiring from the predetermined knowledge graph a probability of occurrence of each of the candidate diseases.

11. The method according to claim 1, wherein before the step of, according to the case-history symptoms and the case-history diseases in the case-history datum, determining from the predetermined knowledge graph the candidate disease that is capable of generating the case-history datum, the method further comprises:
performing entity identification to the case-history datum, to obtain entity references in the case-history datum;
performing entity linking to the entity references in the predetermined knowledge graph, to obtain matched entities in the predetermined knowledge graph of the entity references;
screening out from the matched entities symptom entities that characterize symptoms, to obtain the case-history symptoms of the case-history datum; and
screening out from the matched entities disease entities that characterize diseases, to obtain the case-history diseases of the case-history datum.

12. The method according to claim 11, wherein the step of performing entity linking to the entity references in the predetermined knowledge graph, to obtain the matched entity in the predetermined knowledge graph of the entity references comprises:
for each of the entities comprised by the predetermined knowledge graph, calculating similarities between the entity references and each of the entities; and
linking the entity references to a target entity corresponding to a largest similarity of the similarities, to use the target entity as the matched entity in the predetermined knowledge graph of the entity references.

13. The method according to claim 12, wherein the step of calculating the similarities between the entity references and each of the entities comprises:
for any one of the entities, calculating initial similarities between the entity references and the entity by using at least two similarity calculating modes; and
calculating an average value of the initial similarities that are obtained by calculation, to obtain a similarity between the entity references and the entity.

14. The method according to claim 13, wherein the initial similarities comprise at least two of an edit-distance similarity, a Jaccard similarity, a longest-common-substring similarity, a cosine similarity, an explicit-semantic-analysis similarity and a deep-learning similarity.

15. The method according to claim 11, wherein the step of performing entity identification to the case-history datum, to obtain the entity references in the case-history datum comprises:
performing entity identification to the case-history datum according to a predetermined dictionary comprising a plurality of entity names, to obtain the entity references in the case-history datum.

16. The method according to claim 15, wherein the step of performing entity identification to the case-history datum according to the predetermined dictionary comprising the plurality of entity names, to obtain the entity references in the case-history datum comprises:
according to the predetermined dictionary comprising the plurality of entity names, performing entity identification to the case-history datum by using a bidirectional maximum matching algorithm, to obtain the entity references in the case-history datum.

17. The method according to claim 1, wherein the first weight vector and the second weight vector have equal dimensionalities, the dimensionalities are a quantity of diseases in the preset-disease set, and the step of fusing the first weight vector and the second weight vector, to obtain the disease-analysis vector corresponding to the case-history datum comprises:
weighting the first possibility weight and the second possibility weight with the equal dimensionalities by using different preset importance coefficients, to obtain weighted parameters, wherein a preset importance coefficient corresponding to the first possibility weight and a preset importance coefficient corresponding to the second possibility weight are negatively correlated; and
calculating the weighted parameters by using a linear function or a nonlinear function, to obtain fused weights, wherein the fused weights form the disease-analysis vector corresponding to the case-history datum, and the disease-analysis vector has a dimensionality equal to the dimensionalities of the first weight vector and the second weight vector.

18. The method according to claim 1, wherein the step of performing the target process to obtain the disease-analysis vector corresponding to the case-history datum comprises:
inputting the case-history datum into a predetermined analyzing model, so that the predetermined analyzing model performs the target process, and outputs the disease-analysis vector corresponding to the case-history datum; and
before the step of acquiring the case-history datum, the method further comprises:
acquiring a case-history-datum training set and a case-history-datum test set;
according to the case-history-datum training set and a predetermined loss function, training an original analyzing model, to obtain an intermediate analyzing model; and
testing the intermediate analyzing model according to the case-history-datum test set, to obtain the predetermined analyzing model.

19. An apparatus for predicting a diabetes complication, wherein the apparatus comprises a processor, a memory and a program stored in the memory and executable in the processor, and the program, when executed by the processor, implements the steps of the method for processing medical data according to claim 1, to obtain the disease-analysis vector corresponding to the case-history datum, wherein the preset-disease set comprises one or more diabetes complications, and each of components of the disease-analysis vector represents an illness probability corresponding to each of the diabetes complications.

20. A non-transitory computer-readable storage medium, wherein an instruction in the storage medium, when executed by a processor of an electronic device, enables the electronic device to implement the method for processing medical data according to claim 1.
